(19) United States
(12) Patent Application Publication
(10) Pub. No.: US 2016/0051477 A1
(43) Pub. Date: Feb. 25, 2016

(54) Method for Administering Metformin

(71) Applicant: Saji Gopinathan, Knoxville, TN (US)

(72) Inventor: Saji Gopinathan, Knoxville, TN (US)

(21) Appl. No.: 14/832,232

(22) Filed: Aug. 21, 2015

Related U.S. Application Data
(60) Provisional application No. 62/040,098, filed on Aug. 21, 2014.

(51) Int. Cl. A61K 9/28; A61K 31/155

(52) U.S. Cl. CPC A61K 9/28; A61K 31/155

(57) ABSTRACT

A device and method for administering a consistent and steady dosage of Metformin to patients which includes an implantation component with a composition of Metformin adapted to be inserted either subdermally or subcutaneously to administer a steady incremental dosage of Metformin to the patient throughout a predetermined period of time. A Metformin drug suspension is dispersed in a plurality of microreservoirs and the mixture of microreservoirs and placed in a silicone polymer tube for in situ polymerization and molding, which would allow Metformin molecules to initially diffuse through the microreservoir membrane and then through the silicone polymer containing membrane. In a further embodiment, Metformin implants may be formed by dispersing the drug in a copolymer matrix, which is then coated with a copolymer that serves as a rate-controlling membrane. The drug permeation through the polymer membrane occurs at a rate that is slower than that through the polymer matrix.

### CROSS-REFERENCE TO RELATED APPLICATIONS

This Non-provisional Patent Application claims the benefit of U.S. Provisional Patent Application Ser. No. 62/040,098, filed on Aug. 21, 2014, which is incorporated herein in its entirety by reference.

### STATEMENT REGARDING FEDERALLY-SPONSORED RESEARCH OR DEVELOPMENT

Not Applicable

### BACKGROUND OF THE INVENTION

1. Field of Invention

The present invention relates generally to diabetes medication delivery and in particular to apparatuses, systems and methods for administering a consistent and steady dosage of Metformin to patients.

2. Description of the Related Art

About 23.6 million people in the United States have diabetes, it is a disorder that affects the way the body uses digested food for growth and energy. Normally, the food one eats is broken down into glucose, a form of sugar. The glucose then passes into the bloodstream, where it is used by the cells for growth and energy. For glucose to reach the cells, however, insulin must be present. Insulin is a hormone produced by the pancreas, a fist-sized gland behind the stomach. Most people with type 2 diabetes have two problems: insulin resistance—a condition in which muscle, liver, and fat cells do not use insulin properly—and reduced insulin production by the pancreas. As a result, glucose builds up in the blood, overflows into the urine, and passes out of the body, never fulfilling its role as the body's main source of fuel. If left untreated, diabetes could cause kidney failure, limb amputation, and new-onset blindneness and other life threatening conditions.

One of the most effective ways to treat diabetes is through prevention and timely treatment during the prediabetes stage. Prediabetes is a condition in which blood glucose levels are higher than normal but not high enough for a diagnosis of diabetes. Those with prediabetes are likely to develop type 2 diabetes within 10 years, unless they take steps to prevent or delay diabetes.

One way to treat prediabetes is through the use of the drug Metformin. Insulin produced by the pancreas of pre-diabetic or diabetic patients is not able to get sugar into the cells of the body where it can work properly. Use of Metformin has been shown to lower blood sugar when it is too high and help restore the way a patient metabolizes food in order to make energy. Studies have shown that millions of high-risk people can delay or avoid developing type 2 diabetes by taking consistent dosage of Metformin. Often the treatment time-span to avoid potential development of diabetes is critical and requires consistent commitment from the patient to take the proper dosage of Metformin.

Currently, there are three methods to administer Metformin: oral tablet, extended release oral tablet, and oral solution. All three methods require that patients take Metformin orally at least once a day and on most occasions, twice a day. Children 10 years of age and older are often advised to take it twice a day in smaller doses to ensure safety and minimize the possibility of an overdose. Taking Metformin consistently at the right time is crucial if the medicine is to work in a proper manner. It is also not advisable to crush, break, or chew the oral tablet. For oral liquid solution, patients must measure the oral liquid with a marked measuring spoon, oral syringe, or medicine cup to ensure proper dosage. Serious health issues and complications can arise if a patient either misses a dose or attempts to compensate for a missed dose by taking a double dose. Because Metformin is administered orally, its bioavailability depends on many factors, such as absorption and rate of metabolism in the liver and excretion through the kidney.

The methods currently available for administering Metformin are cumbersome for patients to maintain a consistent and steady dosage schedule, and it is particular difficult for children. Because maintaining a regular dosing schedule is crucial in the treatment of type 2 diabetes and particularly in treatment of pre-diabetic conditions, pre-diabetic children often miss the optimal treatment time due to a lack of steady and consistent intake of Metformin. What is missing in the art is a simple, easy, and less cumbersome method of administering Metformin for patients that offers a consistent effective dosage. Further, and more specifically, what is missing in the art is a method of administering Metformin by means of a subdermal or subcutaneous implant in order to bypass the patient's gastrointestinal tract and eliminate the first-pass effect resulting from metabolism of Metformin in the liver thereby increasing the bioavailability of Metformin and, thereby, reducing the overall dosage that must be administered to the patient.

### BRIEF SUMMARY OF THE INVENTION

Aspects of the present invention disclose a device and method for administering a consistent and steady dosage of Metformin to patients. The administering device includes an implantation component with a composition of Metformin adapted to be inserted under the skin of a patient's arm to administer a steady incremental dosage to a patient throughout a predetermined period of time.

Disclosed herein are apparatuses, compositions, and methods for administering a steady dosage of Metformin to a patient. In some embodiments, a proper dosage of Metformin is administered to a patient in steady increments over a predetermined period of time through an implant that contains a composition of Metformin.

### BRIEF DESCRIPTION OF THE DRAWINGS

The above-mentioned and additional features of the invention will become more clearly understood from the following detailed description of the invention read together with the drawings in which:

FIG. 1 is a perspective view of a device for administering a consistent and steading dosage of Metformin to a patient according to an example embodiment of the present general inventive concept.

FIG. 2 illustrates the drug releasing mechanism according to one example embodiment of the present general inventive concept.

FIG. 3 illustrates the drug releasing mechanism according to another example embodiment of the present general inventive concept.

### DETAILED DESCRIPTION OF THE INVENTION

In accordance with several features of the present general inventive concept, various exemplary embodiments of a drug administering system are disclosed herein and in the accompanying Figures. In several embodiments, the drug delivery system provides an implant assembly having a preloaded dosage of diabetes medication designed to release the medication in a controlled manner once it is inserted into a human body. In an exemplary embodiment, the implant assembly is constructed in a substantially cylindrical shape containing a predetermined dosage of Metformin. When the implant is inserted into a patient's body, it will gradually release the medication over an extended period of time.

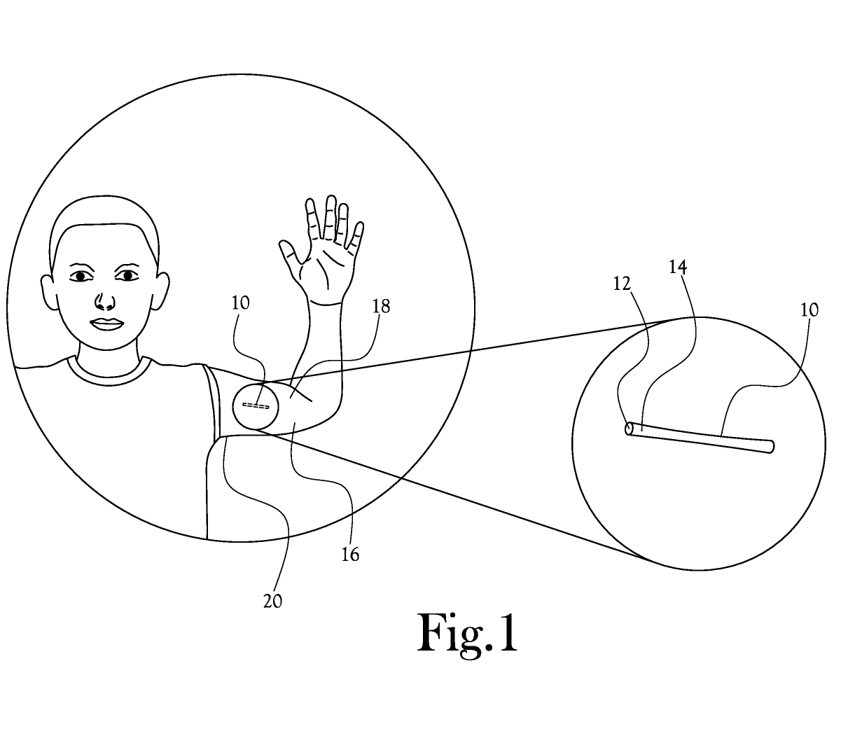

An exemplary embodiment of the drug administering system constructed in according with several features of the present general inventive concept is illustrated in FIG. 1. With reference to FIG. 1, the drug implant 10, containing a diabetes medicine such as, in an exemplary embodiment, Metformin, defines a thin substantially rod shaped device adapted to be inserted subdermally into a human body. However, it will be recognized that other shapes, such as for example spherical, semi-cylindrical and cubical, and the like, may be used without departing from the spirit and scope of the present general inventive concept. The drug implant 10 may include an inner core 12 and a coating membrane 14 wherein the drug is stored in the inner core 12 of drug implant 10.

The drug implant 10, in an exemplary embodiment, is constructed from a generally nontoxic material that is suitable to be inserted subdermally into a human body without creating severe tissue-implant interactions. As illustrated in FIG. 1, the drug implant 10, in an exemplary embodiment, is inserted under the skin into the subdermal tissue on the inner side 16 of the arm between the biceps 18 and triceps muscles 20. Once the drug implant 10 is inserted, the patient does not have to worry about taking the medicine daily. The implant is subtle and should not be considerably noticeable once it is inserted into a patient's body.

In one embodiment, the drug implant 10 is inserted under the skin into the subdermal tissue using a needle-like applicator as will be understood by those skilled in the art. However, it will be recognized that other injection methods may be used, such as for example surgical placement, and the like, without departing from the spirit and scope of the present general inventive concept. Once the drug implant 10 is inserted into the body, the drug implant 10 will remain intact in the body and release a steady dose of diabetes medication, such as Metformin, into a patient's body. The mechanism of action of Metformin may depend on the Metformin activity and dosage. In several embodiments, the drug is released from the implant via diffusion and absorbed into the blood stream.

In an exemplary embodiment, the drug implant 10 containing Metformin allows temporary storage of drug payloads prior to the drug release in the body. Metformin may be released either slowly over time through nanoporous membranes, orifices located in the implant wall, or at precise times when an impermeable seal is removed, to allow drug release by diffusion or expulsion through other transport processes. Metformin has a half-life of around 6 hours, meaning, as will be understood by those skilled in the art, one half of a dose of Metformin is metabolized in approximately 6 hours. Accordingly, in an exemplary embodiment, the implant 10 is adapted to deliver Metformin approximately three to four times a day, i.e. over a twenty-four hour period, in order to give a steady state level of medication in the patient's bloodstream.

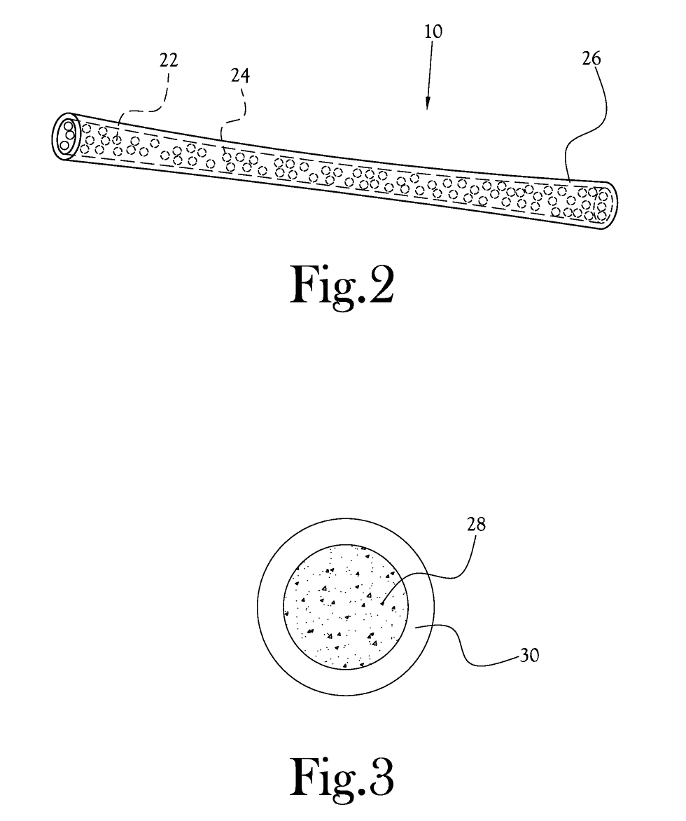

Pursuant to several features of the present general inventive concept, Metformin implants may be placed either subdermally or subcutaneously to control drug release via various drug release mechanisms. In several embodiments, the drug release mechanism may be achieved using reservoir/matrix hybrid-type polymeric implants so that release approximates the constant release from the reservoir system. In one embodiment as illustrated in FIG. 2, a Metformin drug suspension is dispersed in a plurality of individual microreservoirs 22 and the mixture of microreservoirs 22 is placed in a silicone polymer tube for in situ polymerization and molding, which would allow Metformin molecules to initially diffuse through the microreservoir membrane 24 and then through the silicone polymer containing membrane 26. This mechanism allows a consistent release of Metformin over a predetermined duration of time. Furthermore, as illustrated in FIG. 3, in one embodiment, Metformin implants 10 may be formed by dispersing the drug in a copolymer matrix 28. This polymer matrix 28 is then coated with a copolymer, which serves as a rate-controlling membrane 30. The drug permeation through the polymer membrane 30 occurs at a rate that is slower than that through the polymer matrix 28, thus diffusion through the membrane is rate-limiting.

Pursuant to several features of the present general inventive concept, both non-biodegradable polymers and biodegradable polymer may be used to contrast the drug implant 10 to allow a constant and programmed drug released rate for a predetermined duration of medication. It will be understood by those skilled in the art that the drug implant 10 will need to be removed and replaced at regular, selected, intervals. As will be understood, the drug implant 10 is removed by making a small incision the skin, under local anesthesia, over the end of the implant site and grasping the drug implant. The drug implant 10 may then be grasped with forceps and removed from the body.

While the present invention has been illustrated by description of some embodiments, and while the illustrative embodiments have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A method for administrating medication for treating diabetic conditions and pre-diabetic conditions to a patient, comprising:
selecting an implant adapted to be inserted into a patient's body to administer an incremental dosage of the medication to a patient throughout a predetermined period of time;
selecting an appropriate dosage of said medication for treating diabetic conditions and pre-diabetic conditions to be contained within said implant whereby said medication for treating diabetic conditions and pre-diabetic conditions is diffused from said implant and absorbed into the patient's bloodstream; and
implanting said implant at a selected site on the patient's body.

2. The method of claim 1, wherein said implant includes an inner core and an outer coating membrane.

3. The method of claim 1, wherein said implant is constructed with a hybrid formation of reservoir and matrix polymers.

4. The method of claim 1, wherein said implant is adapted to release a selected amount of said medication for treating diabetic conditions and pre-diabetic conditions approximately three to four times over a period of twenty-four hours.

5. The method of claim 1, wherein the method includes the steps of establishing a duration of time that said implant will remain implanted in the patient's body and removing said implant at the end of said duration of time.

6. The method of claim 1, wherein said implants are formed by dispersing the medication for treating diabetic conditions and pre-diabetic conditions in a copolymer matrix, and further wherein the copolymer matrix is then coated with a copolymer, which serves as a rate-controlling membrane, whereby the medication for treating diabetic conditions and pre-diabetic conditions permeates through the copolymer membrane at a rate that is slower than through the polymer matrix.

7. A method for administrating medication for treating diabetic conditions and pre-diabetic conditions to a patient, comprising:
selecting an implant adapted to be inserted into a patient's body to administer an incremental dosage of the medication to a patient throughout a predetermined period of time;
selecting an appropriate dosage of said medication for treating diabetic conditions and pre-diabetic conditions to be contained within said implant whereby said medication for treating diabetic conditions and pre-diabetic conditions is diffused from said implant and absorbed into the patient's bloodstream;
implanting said implant at a selected site on the patient's body;
establishing a duration of time that said implant will remain implanted in the patient's body; and
removing said implant at the end of said duration of time.

8. The method of claim 7, wherein said implant includes an inner core and an outer coating membrane.

9. The method of claim 7, wherein said implant is constructed with a hybrid formation of reservoir and matrix polymers.

10. The method of claim 7, wherein said implant is adapted to release a selected amount of said medication for treating diabetic conditions and pre-diabetic conditions approximately three to four times over a period of twenty-four hours.

11. The method of claim 7, wherein said implants are formed by dispersing the medication for treating diabetic conditions and pre-diabetic conditions in a copolymer matrix, and further wherein the copolymer matrix is then coated with a copolymer, which serves as a rate-controlling membrane, whereby the medication for treating diabetic conditions and pre-diabetic conditions permeates through the copolymer membrane at a rate that is slower than through the polymer matrix.

12. A method for administrating medication for treating diabetic conditions and pre-diabetic conditions to a patient, comprising:
selecting an implant adapted to be inserted into a patient's body to administer an incremental dosage of the medication to a patient throughout a predetermined period of time, wherein said implant is adapted to release a selected amount of said medication for treating diabetic conditions and pre-diabetic conditions approximately three to four times over a period of twenty-four hours;
selecting an appropriate dosage of said medication for treating diabetic conditions and pre-diabetic conditions to be contained within said implant whereby said medication for treating diabetic conditions and pre-diabetic conditions is diffused from said implant and absorbed into the patient's bloodstream;
implanting said implant at a selected site on the patient's body;
establishing a duration of time that said implant will remain implanted in the patient's body; and
removing said implant at the end of said duration of time.

13. The method of claim 12, wherein said implant includes an inner core and an outer coating membrane.

14. The method of claim 12, wherein said implant is constructed with a hybrid formation of reservoir and matrix polymers.

15. The method of claim 12, wherein said implants are formed by dispersing the medication for treating diabetic conditions and pre-diabetic conditions in a copolymer matrix, and further wherein the copolymer matrix is then coated with a copolymer, which serves as a rate-controlling membrane, whereby the medication for treating diabetic conditions and pre-diabetic conditions permeates through the copolymer membrane at a rate that is slower than through the polymer matrix.
